(19) United States
(12) Patent Application Publication
(10) Pub. No.: US 2017/0108509 A1
(43) Pub. Date: Apr. 20, 2017

(54) Method For Using Protein Databases To Identify Microorganisms

(71) Applicant: Virgin Instruments Corporation, Marlborough, MA (US)

(72) Inventors: Kenneth Parker, Hopkinton, MA (US); Marvin L. Vestal, Framingham, MA (US)

(73) Assignee: Virgin Instruments Corporation, Marlborough, MA (US)

(21) Appl. No.: 14/887,281

(22) Filed: Oct. 19, 2015

(51) Int. Cl. G01N 33/68; H01J 49/40; G06F 19/22; C40B 30/02; G06F 19/18; H01J 49/16; H01J 49/00

(52) U.S. Cl. CPC G01N 33/6851; H01J 49/164; H01J 49/40; H01J 49/0036; C40B 30/02; G06F 19/18; G06F 19/22; G01N 2570/00; G01N 2333/245; G01N 2333/25; G01N 2333/26; G01N 2333/255

(57) ABSTRACT

A method for identifying microorganisms by MALDI-TOF mass spectrometry includes acquiring a MALDI mass spectrum of a microorganism, detecting peaks in the acquired MALDI spectrum, generating a peak list comprising mass and intensity from the detected peaks in the acquired MALDI spectrum, acquiring a database of protein sequences deduced from DNA sequences for microorganisms, generating a sub-database of ribosomal proteins from the protein sequences and their masses in the database, matching masses of the detected peaks in the acquired MALDI spectrum to masses of the ribosomal proteins in the generated sub-database, scoring the matches obtained above for each represented microorganism, generating a peak list of accurate masses of matched ribosomal proteins, recalibrating the peak list comprising mass and intensity with the peak list of accurate masses of matched ribosomal proteins, identifying a microorganism with the highest score, and repeating until a desired improvement in the recalibrated peak list or a validated identification is achieved.

The section headings used herein are for organizational purposes only and should not to be construed as limiting the subject matter described in the present application in any way.

### INTRODUCTION

Matrix Assist Laser Desorption Ionization (MALDI) time-of-flight mass spectrometry of intact colonies is currently being used for bacterial colony recognition in clinical environments. Bacterial colony identifications are performed by comparing MALDI time-of-flight mass spectra from individual colonies to mass spectra that have been deposited in libraries, which are derived from many individual isolates. It is known in the art that for some organisms, many signals correspond to ribosomal proteins, which are expressed at high levels. See, for example, Ryzhov, V. and Fenselau, C. (2001), “Characterization of the protein subset desorbed by MALDI from whole bacterial cells”, Anal. Chem. 73, 746-750. Most ribosomal protein subunits are expressed at a 1:1 stoichiometry in the fundamental ribosomal protein translation particle. Moreover, many ribosomal protein subunits have low molecular weights, and are often highly positively charged. See, for example, Arnold et al. 1999 “Monitoring the Growth of a Bacteria Culture by MALDI-MS of Whole Cells”, Anal. Chem. 1999, 71, 1990-1996. Both of these attributes make them readily detectable by MALDI time-of-flight mass spectrometry. The approximate molecular weight of most ribosomal proteins is conserved across all bacteria, and the 60 or so ribosomal protein subunits can be readily identified from DNA sequences of any bacterial species by commonly used bioinformatic tools. They tend to be encoded together in a small number of clusters on the bacterial chromosome. See, for example, Coenye T., et al., (2005), “Advenella incenata gen. nov., sp. nov., A Novel Member of the Alcaligenaceae, Isolated From Various Clinical Samples”, Int. J. Syst. Evol. Microbiol, 55, 251-256. The sequences of many ribosomal protein subunits are often invariant within bacterial species, and conveniently, there is usually a set of substitutions in ribosomal subunits that distinguish species in the same bacterial genus. Most bacterial species also contain some ribosomal protein variation that can distinguish many strains. For all these reasons, ribosomal protein profiling together with MALDI time-of-flight spectroscopy is a powerful method to identify microorganisms.

Three major mass databases have been developed for microbial identification by MALDI time-of-flight mass spectrometry by protein profiling. The masses in these databases have been determined directly from protein extracts, and mostly have not been correlated to protein masses deduced from DNA databases. Currently, two companies, Bruker Corporation and BioMérieux Inc. have received European ‘C’ mark and U.S. Food and Drug Administration approval for the use of their company's database by clinical laboratories. The data in these databases are gathered from bacterial strains that have been collected from human patients, and are focused on clinical goals of identifying disease-causing organisms for determining antibiotic prescriptions as quickly as possible. The National Institute of Health (NIH) also maintains prepared libraries with mechanisms provided for scientists to deposit and retrieve library information of their research interest. However, there has been no systematic effort to expand these library-based methods to include all bacterial species whose genomes have been elucidated so as to enable widespread use and general search capabilities.

### BRIEF DESCRIPTION OF THE DRAWINGS

The present teaching, in accordance with preferred and exemplary embodiments, together with further advantages thereof, is more particularly described in the following detailed description, taken in conjunction with the accompanying drawings. The skilled person in the art will understand that the drawings, described below, are for illustration purposes only. The drawings are not necessarily to scale, emphasis instead generally being placed upon illustrating principles of the teaching. The drawings are not intended to limit the scope of the Applicant's teaching in any way.

FIG. 1 illustrates a method of identifying microorganisms using protein databases according to the current teaching.

FIG. 2 illustrates a plot showing scores for organisms with distinct ribosomal subunit sequences matching to an E. coli spectrum. The rank of each organism is plotted along the x axis using a logarithmic scale to emphasize that the majority of organisms with scores >100 (about 1000 organisms) are related phylogenetically to E. coli.

FIG. 3 illustrates a plot of scores vs. organism rank for the spectrum with and without internal calibration applied.

FIG. 4A and FIG. 4B illustrate two spectra acquired by MALDI spectrometry that map to two distinct clusters of strains of Enterobacter cloacae.

FIG. 5A and FIG. 5B illustrate plots of the scores for forty-two strains of Enterobacter cloacae matching to the spectra in FIGS. 4A and 4B.

FIG. 6A shows a dendrogram of the strains plotted in FIGS. 4A and 4B and FIGS. 5A and 5B.

FIG. 6B illustrates a prior art dendrogram downloaded from http://www.ncbi.nlm.nih.gov/genome/?term=Enterobacter+cloacae.

### DESCRIPTION OF VARIOUS EMBODIMENTS

Reference in the specification to “one embodiment” or “an embodiment” means that a particular feature, structure, or characteristic described in connection with the embodiment is included in at least one embodiment of the teaching. The appearances of the phrase “in one embodiment” in various places in the specification are not necessarily all referring to the same embodiment.

It should be understood that the individual steps of the methods of the present teaching may be performed in any order and/or simultaneously as long as the teaching remains operable. Furthermore, it should be understood that the apparatus and methods of the present teaching can include any number or all of the described embodiments as long as the teaching remains operable.

The present teaching will now be described in more detail with reference to exemplary embodiments thereof as shown in the accompanying drawings. While the present teaching is described in conjunction with various embodiments and examples, it is not intended that the present teaching be limited to such embodiments. On the contrary, the present teaching encompasses various alternatives, modifications and equivalents, as will be appreciated by those of skill in the art. Those of ordinary skill in the art having access to the teaching herein will recognize additional implementations, modifications, and embodiments, as well as other fields of use, which are within the scope of the present disclosure as described herein.

The methods of the present teaching can use data generated by a MALDI-TOF mass spectrometer. Recent developments in MALDI-TOF mass spectrometry, which are described in U.S. Pat. No. 8,735,810, entitled “Time-of-Flight Mass Spectrometer with Ion Source and Ion Detector Electrically Connected,” U.S. patent application Ser. No. 14/462,146, entitled “Ion Optical System for MALDI-TOF Mass Spectrometer,” and U.S. Provisional Application Ser. No. 62/139,889, entitled “Mass Spectrometry Method and Apparatus for Clinical Diagnostic Applications,” describe mass spectrometers that produce MALDI-TOF mass spectra where both the intensity and the mass of peaks in the spectra are highly reproducible and, therefore, work particularly well with the methods according to the present teaching. U.S. Pat. No. 8,735,810, U.S. patent application Ser. No. 14/462,146, and U.S. Provisional Application Ser. No. 62/139,885 are all assigned to the present assignee and the entire contents of this patent and these patent applications are herein incorporated by reference. High quality MALDI TOF mass spectrometers that incorporate the improvements described in this patent and these patent applications effectively reduce variability in results due to instrument imperfections and heterogeneity in sample preparations to the point that the effects are negligible in the quality of the results obtained.

The present teaching relates to methods that use proteome information derived from DNA sequencing to enable organism identification by MALDI, a technique we refer to herein as RiboPMF. The method of the present teaching makes it possible to attempt bacterial identification of any species that has been completely sequenced, and proposes protein sequences for many of the peaks in each mass spectrum, which can be verified, if desired. There are no limits on the number of bacterial strains that can be screened by the methods of the present teaching. At least 20,000 bacterial strains can be searched in a few minutes without any serious effort to optimize search speed using a desk-top computer.

A method for identifying microorganisms by MALDI-TOF mass spectrometry according to the present teaching includes acquiring a MALDI mass spectrum of a microorganism. Peaks in the acquired MALDI spectrum are detected. A peak list comprising mass and intensity from the detected peaks in the acquired MALDI spectrum is then generated. A database of protein sequences translated from DNA sequences for microorganisms is downloaded from public web sites, or generated in-house from whole organism DNA sequencing. From these databases, a sub-database of ribosomal proteins from the DNA sequences and their masses is generated. Masses of the detected peaks in the acquired MALDI spectrum are matched to masses of the ribosomal proteins in the generated sub-database. The match between the mass spectrum and the ribosomal proteins predicted for each microorganism represented in the database is scored according to the percentage of intensity in the peak list that is matched (% I), the percentage of ribosomal proteins that can be accounted for (% R) that have masses in the appropriate mass range for the spectrum, and the intensity-weighted average mass error (ppm) for the matches. A peak list of accurate masses of matched ribosomal proteins is then generated. The mass peak list is recalibrated with the peak list of accurate masses of matched ribosomal proteins if necessary. The matching masses of the detected peaks and the scoring of the match between the mass spectrum and the predicted ribosomal proteins using the recalibrated peak list is then repeated to improve and validate identification until a desired identification is achieved.

The methods of the present teaching are useful for clinical medicine because they simplify the task of explaining why the library methods work by proposing specific protein sequences for each observed mass. The method of the present teaching is likely to be particularly useful in identifying organisms that are encountered infrequently in clinical settings, or that derive from patients in poorly studied parts of the world, from veterinary samples, or from samples isolated from the environment, such as samples from lakes, oceans, fields, soil, forests, or elsewhere in the lithosphere. At present, the definition of the boundaries of many bacterial species is in a state of flux as more information becomes available. The methods of the present teaching may turn out to useful in defining bacterial species, because ribosomal proteins are among the least variable of proteins in bacteria.

In addition, the methods of the present teaching extend organism identification in several ways. First, in cases where bacterial proteome information is collected, the organisms in question can potentially be identified, even if the organism has never been found previously to be pathogenic, and is therefore not represented in databases derived from extracts of pathogenic organisms.

Second, and on the other end of the pathogenicity scale, there have been gaps in the libraries of pathogens for bioterrorism organisms for the reason that the companies that prepare the libraries do not have the security clearance to grow the organisms to gather spectra for the libraries. These gaps can be filled by methods according to the present teaching, because the DNA sequences and translated protein sequences for these organisms is publicly available.

Third, most library spectra have been gathered from bacteria that infect people from developed countries, yet there is much more bacterial diversity in the tropics, and other less-developed areas of the world. The methods of the present teaching allow expansion to these bacterial species. Because of advances in DNA sequencing, it is likely that in the near future, information for additional organisms will be sequenced much more rapidly and easily than new information can be deposited into the existing extract-based mass databases. Moreover, DNA sequence information is available for certain organisms that cannot be grown in culture, or have not yet been grown in culture.

Fourth, bacteria continually evolve as new ecological niches are created by humans. Prior art library methods will be able to identify newly evolving threats only after they have been found to be a problem. Because the methods of the present teaching identify organisms together with taxonomic tree information, each of the proposed identifications comes along with information about how the organism evolved. Ambiguous identifications are readily apparent when the top scoring organisms do not belong to a taxonomic clade.

Finally, there are many other scientific disciplines other than clinical medicine where identifying bacterial species is important. These scientific disciplines include, for example, veterinary medicine, agriculture, and ecology. We note that until every taxonomic unit is directly assessed, the methods of the present teaching may fail for certain taxonomic categories of bacteria. For example, some gram-negative bacteria are much more easily identified than staphylococci, from which a smaller percentage of peaks correspond to easily predicted ribosomal subunits, using certain preparation methods. It is possible that some bacterial taxa will turn out to significantly harder to identify than staphylococci.

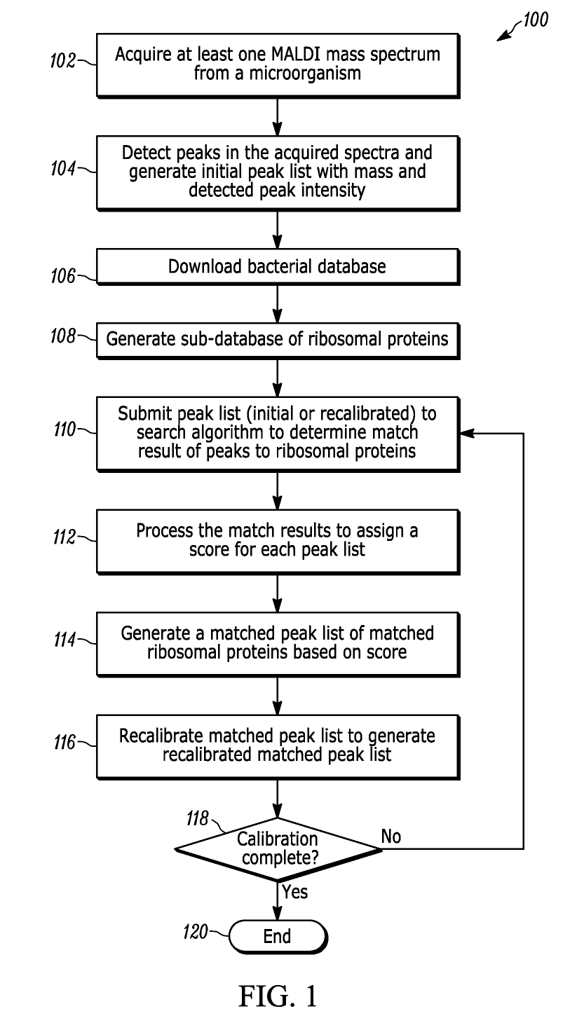

FIG. 1 illustrates an embodiment of the method of identifying microorganisms using protein databases 100 of the current teaching. One skilled in the art will appreciate that the methods according to the present teaching can be implemented by one or more computers. In particular, one skilled in the art will appreciate that one computer can be used to perform all the steps of a method according to the present teaching or different computers can be used to perform one or more steps of a method according to the present teaching. The first step 102 is to acquire one or more mass spectra from an isolate of a microorganism using MALDI-TOF spectrometry. The second step 104 is to detect peaks in each spectra and generate an initial peak list including mass and intensity of detected peaks for each spectra. In some embodiments, an intensity parameter is calculated from the signal-to-noise ratio of the detected peaks in the spectra, or a function of that ratio, such as the signal-to-noise ratio squared is calculated. The output of the second step is one or more peak lists. In some methods according to the present teaching there is one peak list for each MALDI spectrum.

Also, in some methods of the present teaching, the peak list to be matched is optionally filtered to include only pairs of single and doubly charged masses, or where such pairs are weighted higher than the remaining peaks. This follows from the observation that such pairs are more likely to be reproducible.

In the third step 106 of the method, a protein database is downloaded from a public site, which was originally translated from DNA sequence databases. A feature of the present teaching is that it is compatible with standard bioinformatics databases under development by leading scientific organizations. The source for this database may be a public site or a private database. One of the standard bioinformatic databases available to the world's scientist community has been assembled by the UniProt Consortium, www.uniprot.org, which is made up of the European Bioinformatic Institute (EBI) and the European Molecular Biology Laboratory (EMBL). The mission of UniProt is to provide the scientific community with a comprehensive, high-quality and freely accessible resource of protein sequence and functional information.

The UniProt consortium prepares two protein databases, the SwissProt database, with well-studied proteins, and the TrEMBL database, which is much larger, and contains nearly complete proteomes of about 20000 bacterial isolates, many of which are poorly represented in SwissProt. Both the SwissProt and TrEMBL databases are well-annotated regarding ribosomal protein subunits, and are continually being improved. Each organism in both databases is mapped to a taxonomic tree containing the latest knowledge regarding the exact position of the organism within the major taxonomic divisions of bacteria. Such information is also readily available from the National Center for Biotechnology Information (NCBI), but so far we have found it takes fewer steps to download the relevant information from UniProt.

Some embodiments of the method of the present teaching deposit information about all ribosomal protein subunits from TrEMBL, together with taxonomic information, into a relational database. For example, the relational database SQLite3, http://www.sqliteexpert.com/, may be used. Other embodiments of the method of the present teaching deposit all protein sequences into the database deriving from SwissProt alone, or from both SwissProt and TrEMBL. Many embodiments of the present teaching utilize the complete proteome database. In these embodiments, the complete proteome database is much larger. In order to save searching time, some methods query appropriate taxonomic subdivisions of the database rather than search all entries. As such, embodiments that utilize the complete proteome database are most useful for identifying strains after a species or genus has already been identified so that it is not necessary to search the entire the database for all species and genera.

In some embodiments, step three 106 includes downloading the TrEMBL bacterial database from the Expasy site. The source for the database is: ftp://ftp.uniprot.org/pub/databases/uniprot/current_release/knowledgebase/taxonomic_divisions/uniprot_tremb1_bacteria.dat.gz. As of Dec. 1, 2014, this database included 34.8 GByte of data. Alternatively, a similar database of strains and bacterial proteins may be downloaded from NCBI, or another repository of such data.

The fourth step 108 of the method is to generate a sub-database of ribosomal proteins from the bacterial database downloaded from the source in step three 106 using a computer. In some embodiments, the sub-database is generated using c sharp programming. The sub-database of ribosomal proteins is made in SQLite3. SQLite is a C library that provides a database that doesn't require a separate server process. The SQLite3 module provides a DB-API 2.0 compliant interface for SQLite databases. In some methods, the fourth step 108 is best executed overnight because of its computational intensive nature. By generating sub-databases, it is possible to reduce the file size. As an example, generating a sub-database produced a much smaller 86.2 MB file. Furthermore, the generation of sub-databases can combine together bacterial strains that have the exact same set of ribosomal protein subunits. These identical strains cannot be directly distinguished by the method of the present teaching, and so combining identical strains eliminates redundant processing. For example, the Dec. 1, 2014 TrEMBL database has 837 Staphylococcus aureus strains with identical sets of ribosomal protein sequences. Eliminating the identical strains in this example reduces a database with 20,000 strains to a database with only 11,409 strains.

In some methods according to the present teaching, the relational protein sequence sub-databases are prepared from protein databases in which combinations of strain information, sequence information, and/or protein annotation are used to prepare the database. Also, in some methods, sub-databases are prepared when ribosomal protein sequences are specifically extracted from the large database. In still other methods, protein sequence databases are reduced by exact sequence identity to the smallest possible list of distinct sequences, retaining aggregate information like number of identical sequence and taxonomic breadth. In some methods, such aggregated sequences are sorted by percent homology, using any set of adjacent amino acids as an alignment key.

The fifth step 110 of the method includes submitting a peak list to one or more search algorithms executing on a computer to produce a match result of submitted peaks to ribosomal proteins in the database. The peak list can be an initial or a recalibrated peak list. In some embodiments, the fifth step 110 includes searching the SQLite3 database of 11409 organisms to generate match results for the peak list submitted for each spectrum acquired by MALDI mass spectrometry. Also, in some embodiments, each spectrum may contain between twenty and several hundred peaks, together with intensities, with masses anywhere between 2,000 amu and 30,000 amu. It is possible to restrict the mass range further, for example, between 5,000 and 6,000, as a test of the robustness of the identification. Currently, this kind of search takes ˜40 seconds for the ribosomal database with a modern computer, depending on the size of the peak list. Much of this time is spent in reading the ribosomal database into memory, a step which needs to be performed only once if multiple spectra are to be searched. It has been determined that this method appears to work best on spectra that contain a large number of well resolved peaks, for example, on the order of 50-300 peaks.

In one embodiment of the method of the present teaching, the user selects an upper limit for the mass error of the peak matches. For example, this upper limit can be in parts per million (ppm); for example, 1500 ppm. All peak matches between each spectrum and organism are tabulated, along with the percentage of ribosomal proteins identified, the percentage of the intensity in the spectrum that is accounted for by those matches, and the intensity-weighted average ppm accuracy of those matches. In later steps of the method, which are described further below, an overall score is calculated from these statistics, and the table is sorted by the score. It is commonly observed that related organisms commonly receive similar scores. This result is expected because related organisms commonly share many identical ribosomal protein masses and sequences.

In the first pass through the steps of the method 100 of FIG. 1, the submitted peak list is the initial peak list generated in step two 104 from the MALDI mass spectrum acquired in step one 102. In subsequent loops through the steps of the method 100 of FIG. 1, the submitted peak list may be a recalibrated peak list generated in later steps of the method. If calibrants had been added to the sample to improve the beginning mass calibration of the instrument, these calibrant masses can be used as an additional constraint during recalibration.

The results of the search in the fifth step 110 are sensitive to peak detection settings that are selected by the user. It has been determined that the method of the present teaching typically work well with a wide set of parameters. With some internal calibration, and a corresponding reduction in tolerances (for example from 1500 to 250 ppm), the method 100 still yields the correct results even with a peak list containing 500 masses. Large peak lists slow down the duration of the matching process to durations on order of 55 seconds using modern computers. Large peak lists appear to be useful so long as most of the peaks correspond to reproducible spectrum features. The match results produced by the fifth step 110 are then processed in step six 112.

In a sixth step 112 of the method 100, computer processing is used to determine a score for each match result. Every match result from the peak list corresponding to each mass spectra is given a score for each ribosomal protein in the database. Matching statistics are generated and provided to the user in various forms and formats for various embodiments. In contrast to commonly used methods for scoring matches where the count of peaks that are matched is the primary scoring parameter, in methods according to the present teaching other parameters are used to calculate the score. In many methods according to the present teaching the score is calculated as described below taking into account peak intensity, mass accuracy, and differential protein weighting factors that enable correct organism identification. In some methods according to the present teaching, three parameters are calculated using the match result, and these parameters contribute to the overall score for that match result. The first of the three parameters is the percentage of intensity in the peak list that is matched. The second parameter is the percentage of ribosomal proteins that has been matched. One can calculate the percentage of ribosomal proteins that can be accounted for using the actual number of ribosomal proteins listed, or by assuming that there ought to be a number, N, of ribosomal proteins in the mass range of interest (including both singly and doubly charged proteins) so as to avoid favoring strains that are missing annotations to certain subunits. In one specific method, setting N to 80 is effective for searching proteins with masses between 3-16 kDa. For each pairing of sample and organism, the value for N can be adjusted to correspond to the number of ribosomal proteins with masses within a narrow mass range that may be appropriate for that particular spectrum, as in certain cases the mass range that is detected is dependent on sample preparation, which may be sample-specific. In various embodiments, N is species-dependent because certain species contain multiple and different forms of certain ribosomal subunits. When the entire proteome is being searched, the user has the option of weighting the value of each match according to the protein family. For example, one can choose to increase the weight of all ribosomal proteins by 10-fold compared to other proteins. If this is done, strains will be ranked primarily on the basis of ribosomal proteins, yet matches to all other proteins will be displayed, and can still contribute to strain differentiation. As described below, other factors pertaining to individual protein can also be used to adjust the weighting factor for each protein.

The third parameter calculated during the processing in the sixth step 112 for each of the match results provided by the fifth step 110 is the intensity-weighted average mass error (ppm) for the matches. Using these three parameters, the raw score is calculated as:

$$\frac{\begin{matrix}percent intensity matched * \\ percent ribosomal proteins accounted for\end{matrix}}{average mass error in ppm}$$

In some embodiments, the score is processed as the log₁₀ of the parameters in the previous equation, in particular the score can be expressed as:

Score=log₁₀(% R)+log₁₀(% I)−log₁₀(ppm)

where % R is percent of ribosomal proteins matched, % I is the percent total intensity matched, and ppm is the root mean square, RMS, error (in parts per million) of the matched proteins.

In some embodiments, one of the terms is given a higher weighting; for example:

Score=2*log 10(% R)+log 10(% I)−log 10(ppm).

Various embodiments of method of the present teaching provide various presentations of the data to the user. In some methods, for each MALDI spectrum peak list submitted, the score for each ribosomal protein is presented, ranking the particular protein based on its score. In some embodiments, the sixth step 112 also processes the match result by counting how protein species were matched and reports exactly which proteins were matched both by name and by protein sequence. This part of the sixth step processing results is not used in the overall score, but may be presented as data to the user.

In a seventh step 114 of the method 100, some embodiments of the method of the current teaching automatically generate a calibration file of protein masses from the top hit. This calibration file can be used to test the spectrum for internal consistency, if desired. It can also be combined with the masses for calibrant substances that have been added to the sample. If there are multiple spectra identified from the same isolate, the same calibration file should increase the score for each spectrum. In addition, narrowing the mass tolerance should increase the discrimination between the highest scoring species. All other species in the database should increase to a maximum score depending on the internal mass accuracy (i.e. mass error) of the spectrum. Successful calibration should in most cases result in a high number of monomer/dimer pairs within the spectrum with tight tolerance. This is true whether the spectrum is mapped to ribosomal proteins or not.

Thus, the seventh step 114 of the method 100 generates a new list that contains the set of peaks from the protein sequence that had the highest score. In the first pass through the method, this new list is generated for each of the submitted peak list from each mass spectra generated by MALDI spectrometry of an organism. In subsequent passes through the method, this new list is generated for each submitted peak list. The new list represents a calibration file. Thus, in seventh step 114, every time a search is done (successful or not), it generates a calibration file based on the matched ribosomal proteins from the top hit, optionally in combination with mass calibrants. This calibration file may be used in subsequent steps to improve the calibration, thereby making it possible to reduce the mass tolerances.

The eighth step 116 of the method 100 recalibrates the matched peak list the calibration file generated in step seven 114. Following recalibration, the scores for all organisms in the database are recalculated, and sorted to identify the organism with the highest score. In the case of a robust identification, the highest scoring organism is likely to be the same organism that had the highest score prior to internal calibration. There may be more separation by score from alternative organisms, especially from organisms with unrelated ribosomal protein sequences.

The method 100 then moves to a decision point 118, which determines whether the identification method is complete. If so, the method ends, and resulting data is presented to a user for further analysis. If additional calibration or validation is required, the process proceeds back to the fifth step 110. In this case, the submitted peak list is the recalibrated peak list that was generated in the eighth step 116. The recalibrated peak list is submitted to the search algorithm against the database in the fifth step 110, and a match result is obtained. The match result is subsequently processed to assign a score in the sixth step 112. A new matched peak list is generated using the peak list from the ribosomal protein with the highest score in seventh step 114 and this new matched peak list becomes the calibration file. The submitted peak list is recalibrated using the calibration file in the eighth step 116, generating a recalibration file.

The method 100 continues until terminated at the end step 120 based on if the desired improvement in the recalibrated file is achieved and/or identification is validated. In some embodiments, an identification is validated when the highest score achieves a predetermined value. The method 100 generates results that are presented to a user for further analysis and identification. In some embodiments, the results of the method of the present teaching include the computed relative probability that a MALDI-TOF mass spectrum corresponds to an identified microorganism.

In some embodiments of the present teaching, additional proteins, for example DNA binding protein HU, are added to the set or ribosomal proteins to be matched. In some embodiments, homologs, or other proteins found to be important, are added to the sub-database of ribosomal proteins. In some embodiments, the set of proteins to be matched includes doubly charged forms of each protein. In some embodiments, certain proteins, including certain ribosomal proteins, have adjusted molecular weights to account for known stoichiometric modifications like methylation. For example, it appears that E. coli ribosomal protein L11 is methylated. L11 is widely conserved across bacteria (62% homology between E. coli and S. aureus). In some embodiments, certain proteins annotated as ribosomal are decremented in weighting because they are not well conserved across taxa, suggesting they are not the active ribosomal protein species. For example, some bacterial proteomes contain a L11 species that is much less homologous to other L11 molecules in related clades. It is possible that the less homologous L11 molecules are non-functional or contain sequencing errors, and therefore they should be weighted less than usual in deducing strain identity.

The methods of the present teaching have been able to identify every organism from every spectrum starting from the complete set of organisms so long as that same spectrum can be successfully identified using the library approach, with the caveat that it helps to calibrate the mass spectrum first. On one particular plate, with careful choice of peak detection parameters and starting calibration, all fifty-six spectra were correctly identified, starting from calibration on one of the E. coli standard spectra, with the matching tolerance set at 1000 ppm. In general, gram-positive organisms receive lower scores, as spectra from them sometimes have fewer well-defined peaks, as well as many intense peaks that do not correlate to unmodified ribosomal proteins.

One feature of the present teaching is that various presentations and analysis of the matching results can be used to identify organisms, improve the database, and observe features, similarities and differences amongst various species. In some methods according to the present teaching, a score for each organism is presented for each MALDI spectrum peak, ranking the particular organism based on its score.

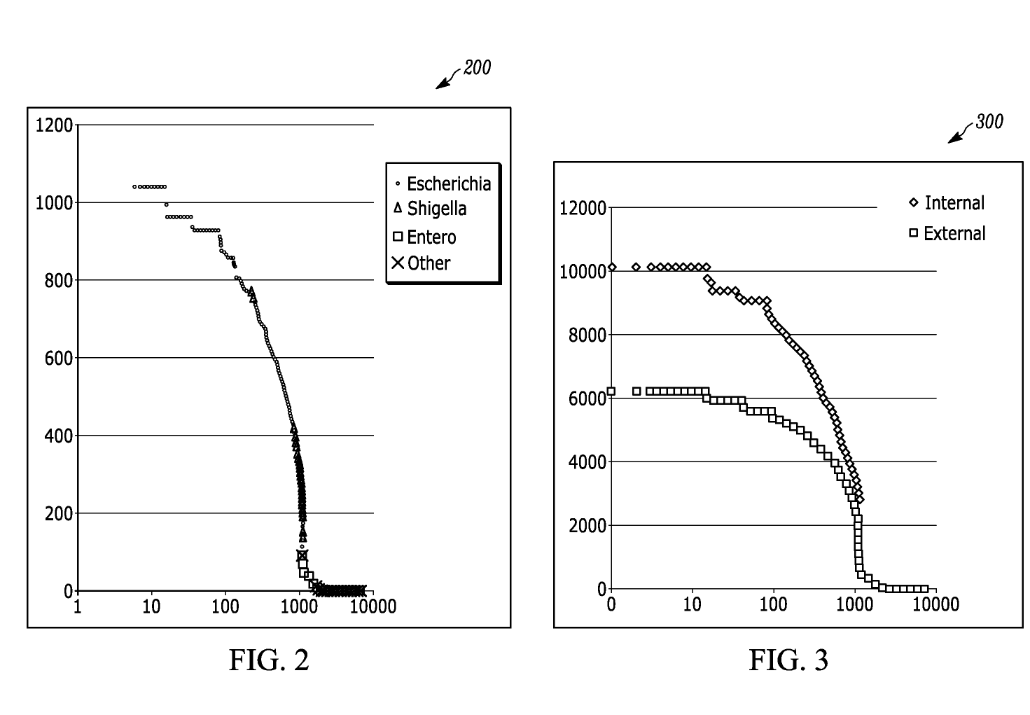

FIG. 2 illustrates a plot 200 of the scores for the 7160 organisms with distinct ribosomal subunit sequences matching to an E. coli spectrum as a function of the rank for a method according to the present teaching. The highest organism score is 10378. Forty-eight of eighty-one ribosomal proteins were matched, accounting for 45% of the total intensity of peaks in the spectrum. There are plateaus of organisms that have identical scores, because they have an identical set of matched ribosomal proteins. The top two hundred and twenty-six proteins are all annotated as E. coli, before the highest scoring entry annotated as Shigella flexneri. The highest scoring organism that is neither E. coli nor Shigella is a Klebsiella strain at rank 1131. In FIG. 2, the organisms are shown in the graph with different symbols according to four categories: E. coli, Shigella, other members of the Enterobacteriaceae (including Klebsiella), and all other organisms.

One feature of the present teaching is the ability to identify an organism in spite of annotation irregularities in the protein sequence database. Some of the differences seen in FIG. 2 relate to scoring within a species like E. coli are due to annotation irregularities of ribosomal proteins, including cases where certain ribosomal proteins are absent altogether, either because they appear to have damaged reading frames (with stop codons), or because they were not correctly recognized by the automatic protein annotation process. Another common annotation error involves extra sequences N-terminal to the accepted start codon, or deletions of the mature N-terminus, often starting at an internal methionine. The extent of these mis-annotations can be determined by monitoring the length of the ribosomal subunits. Thus, one feature of the present teaching is that, until these annotation issues are addressed, the methods of the present teaching have the ability to not only identify the best organism match to a spectrum, but they also identify the strain that has the best annotations that happen to be detectable by MALDI.

One feature of the methods of the present teaching is that they are independent of whether or not the organisms have been correctly annotated with regard to appropriate species, genus, or higher taxonomic classification. The identity of any strain that has scores inconsistent with related strains can be readily verified by examining protein homology starting from the sequence of any of the ribosomal subunits that have been matched. There appear to be organism entries in the databases that are mapped to taxa that are inconsistent with other organisms mapped to the same taxon. This finding indicates that the organism has been misidentified, which would be misleading if the scores of all related organisms were not readily available.

Like all other identification methods, the methods of the present teaching provide no guarantee that the correct answer is in the database. Lower quality spectra generally result in lower scores. Each organism has a maximum score that is unknown to start. As with known methods, sample preparation protocols impact this maximum score. Also, the score is a function of the percentage of intensity in the spectrum that can be accounted for by ribosomal proteins, and the percentage of ribosomal proteins whose masses (both singly charged and doubly charged) that can be distinguished from among the peaks in the spectrum.

Any preparation process that increases MALDI detection of ribosomal proteins in general should increase the score, whereas any process that selectively decreases recognition of ribosomal proteins should decrease the score. We have found it useful to filter the identification list to include strains that have between forty and seventy distinct ribosomal proteins. If a strain contains fewer proteins than forty, some ribosomal proteins are missing, which could lead to misleading results. Similarly, a “strain” with more than seventy ribosomal proteins often appears to be an amalgamation of multiple distinct strains, with multiple polymorphic variant sequences for certain ribosomal proteins.

Sometimes bacterial strains contain more than one gene for a particular ribosomal protein, for example, ribosomal protein L33 in staphylococci. In this situation, the score for these strains might be improved by calculating what percentage of the named ribosomal subunits are accounted for, instead of weighing the singly and doubly charge form of each ribosomal protein independently.

In some methods according to the present teaching, a check for known chemical modifications to ribosomal proteins (like methylation or ribosomal subunit S33, acetylation, or violation of the canonical N-end rule) is performed. Also, in some methods, identification is improved by mapping other well conserved bacterial proteins, for example, the histone-like DNA binding proteins, cold shock proteins, glutaredoxin, ATP synthase epsilon subunit, etc. Some of these proteins have been proposed to account for observed MALDI peaks. See, for example, Ryzhov, V. and Fenselau, C. (2001), “Characterization of the protein subset desorbed by MALDI from whole bacterial cells”, Anal. Chem. 73, 746-750. Also, some methods according to the present teaching assign matches to certain ribosomal proteins a higher weight.

It is possible to perform the same scoring method starting from different databases. For example, one can start from the SwissProt database, and map to every protein. However, many important pathogens are not yet included in SwissProt. For some bacteria and from carefully calibrated high quality spectra with at least one hundred peaks, we have shown that is possible to correctly identify species within a larger clade by searching for the masses of every annotated protein in the database (usually 3000-6000 proteins, with several thousand candidate masses in the region of interest). One disadvantage of including all proteins is that it takes much longer to perform the method. Another disadvantage of including all proteins is that it may also be unsuccessful for poor quality spectra.

To determine the ability of the method of the present teaching to distinguish organisms with similar ribosomal proteins, matching is performed on known Shigella isolates, which share many sequences with E. coli. Organisms that are annotated as Shigella are not monophyletic with respect to E. coli. See, for example, Lan (2004), “Molecular Evolutionary Relationships of Enteroinvasive Escherichia Coli and Shigella spp.”, Infect Immun.; 72:5080-8. Instead, certain clades of E. coli have pathogenicity factors that have been transmitted between strains horizontally rather than vertically, making it difficult to predict how well matching to ribosomal proteins alone ought to be able to separate Shigella from E. coli.

One feature of the present teaching is the use of internal calibrations that are computed with a computer to improve the matching scores and to enhance identification. In some methods according to the present teaching, all proteins in the proteome are considered for matching purposes, where the goal is to differentiate strains. These methods require careful internal calibration.

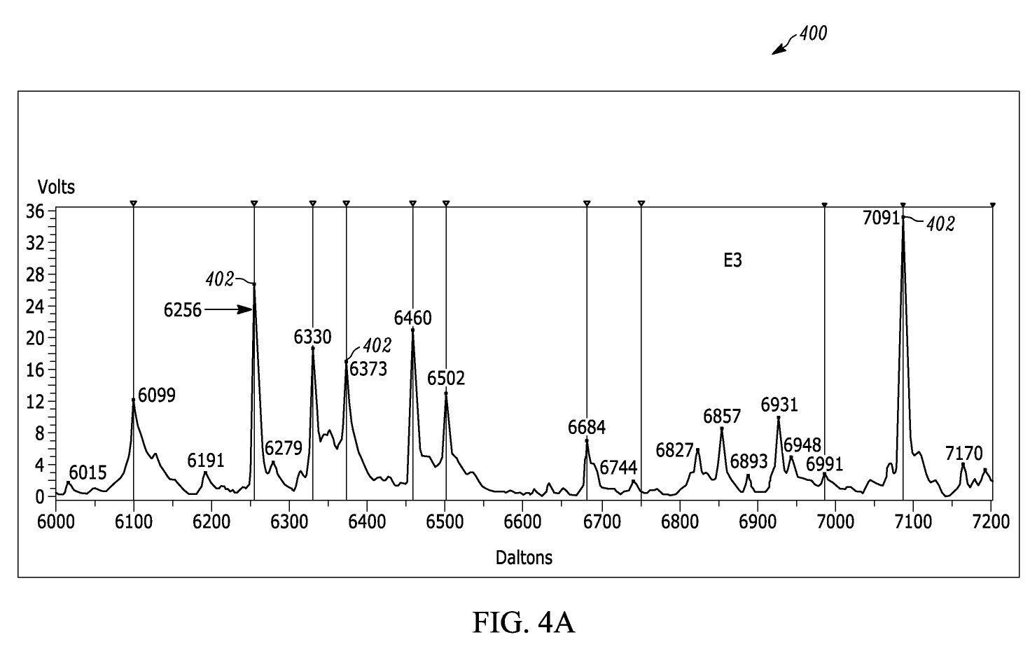

FIG. 3 illustrates a plot 300 of score vs. organism rank for the same spectrum with and without internal calibration applied. It was determined that when the ppm tolerance was set to 250 ppm, peaks matched better after internal calibration, causing the scores to increase from 6226 to 10378. This occurred at least in part because the ppm error term was reduced from 91 ppm to 63 ppm. If the ppm tolerance is set much looser, for example to 1250 ppm, then the pattern of scores is very similar, but there is less ability to distinguish between closely related strains. In the data shown in the plot 300 FIG. 3, the starting calibration was relatively good. In methods using E. coli where a large number of ribosomal proteins are intense, the method of the present teaching returns mostly correct identifications (one aberrant Shigella strain and 1 aberrant Salmonella strain) even with the mass tolerance set to 25,000 ppm starting from 176 peaks.

One embodiment of the method of the present teaching calculates the probability that two strains are indistinguishable by ribosomal protein matching. In some applications, the database contains many organism/strain entries that have identical or nearly identical ribosomal protein sequences. In these applications, it is more difficult to determine how much strain differentiation is possible based solely on ribosomal protein sequences.

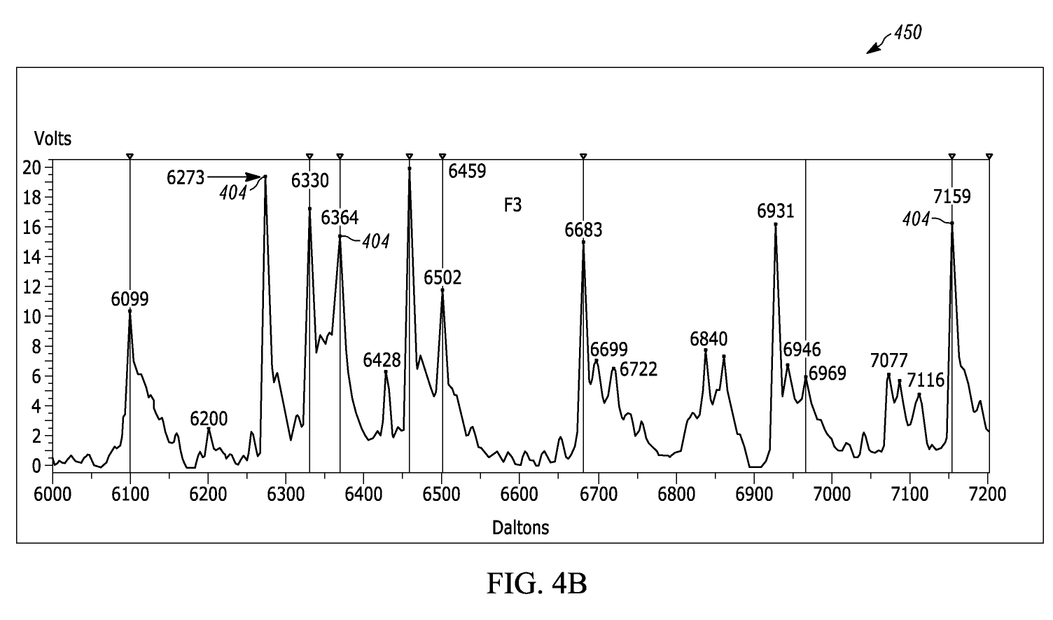

FIG. 4A and FIG. 4B illustrate two spectra 400, 450 that map best to non-overlapping sets of strains of Enterobacter cloacae using a wide range of search criteria. When this happens, the strain differentiation is more credible and can be termed ‘robust’. The spectra 400, 450 illustrated in FIG. 4A and FIG. 4B show only the region between mz 6000 and 7200, but there are differences throughout the 2,000-20,000 mass region. The spectra are overlaid on line diagrams corresponding to all of the masses of ribosomal proteins that match within 250 ppm to the spectrum shown. The arrows 402 and 404 point to ribosomal subunit polymorphisms between the highest matching strains. In FIG. 4A, the matching strains of E. cloacae encode ribosomal protein L33 (methylated) at mz 6256, ribosomal protein L18 (doubly charged) at mz 6373, and ribosomal protein L35 at 7091. For a different set of strains of E. cloacae to which the spectrum in FIG. 4B match, the corresponding ribosomal proteins are expected at mz 6273, 6364 and 7159, respectively. For these three ribosomal proteins, the differences in masses are due to polymorphisms in the designated subunits rather than annotation artifacts.

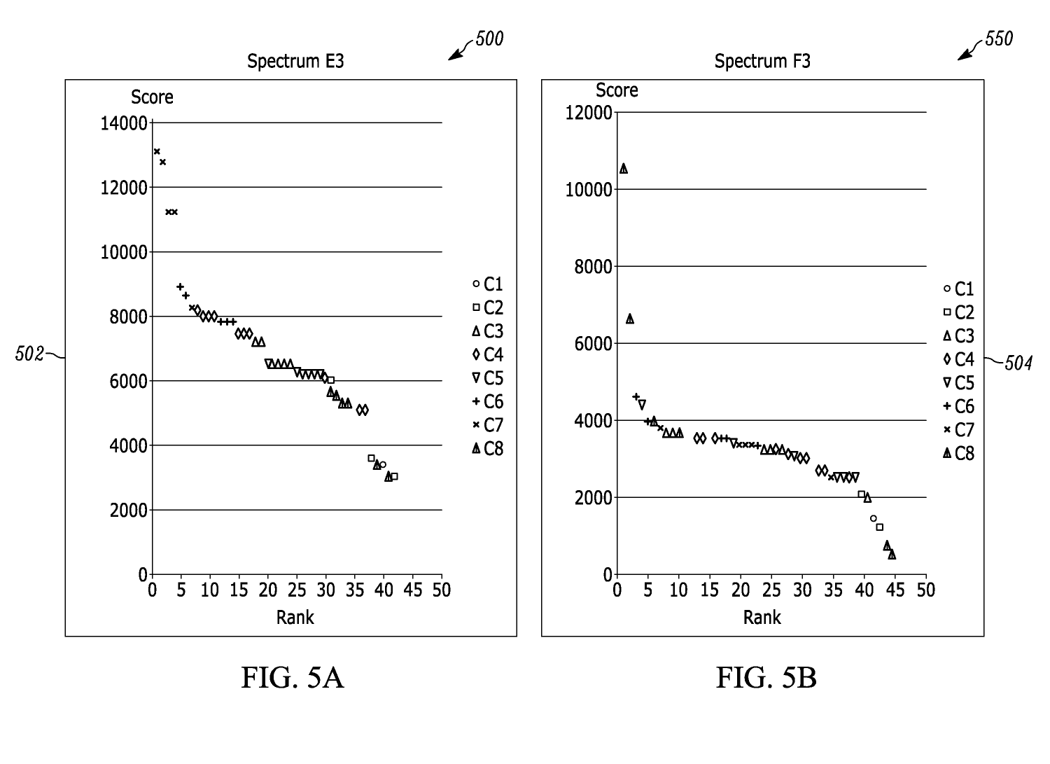

FIGS. 5A and 5B show how the scores of forty-two strains of Enterobacter, divided into 8 clades according to patterns of shared ribosomal protein sequences, are distributed from spectrum E3 (in FIG. 4A) vs. spectrum F3 (FIG. 4B). The different clades are plotted using different symbols on the graph so that the relative ranking of the clades can be visualized. Note that four of the five strains marked using x's from clade 7 score higher than 10,000 for spectrum E3, whereas these same strains receive scores in the 2-4,000 range from spectrum F3. In contrast, two strains from one clade 8 appear to match best (scores 10,513 and 6620) to spectrum F3, whereas these same strains appear to have low scores when matched to spectrum E3 (3350 and 664).

One feature of the method of the present teaching is the ability to easily generate diagrams that show taxonomic relationships based on similarity of ribosomal proteins. To determine how much strain differentiation is possible, the user may generate a table that counts the number of identical ribosomal protein sequences that are shared among the top N database hits. The generated table can then be submitted to hierarchical clustering in R to generate dendrograms that show which strains have the most similar ribosomal profiles. This procedure was used to propose the 8 clades described above.

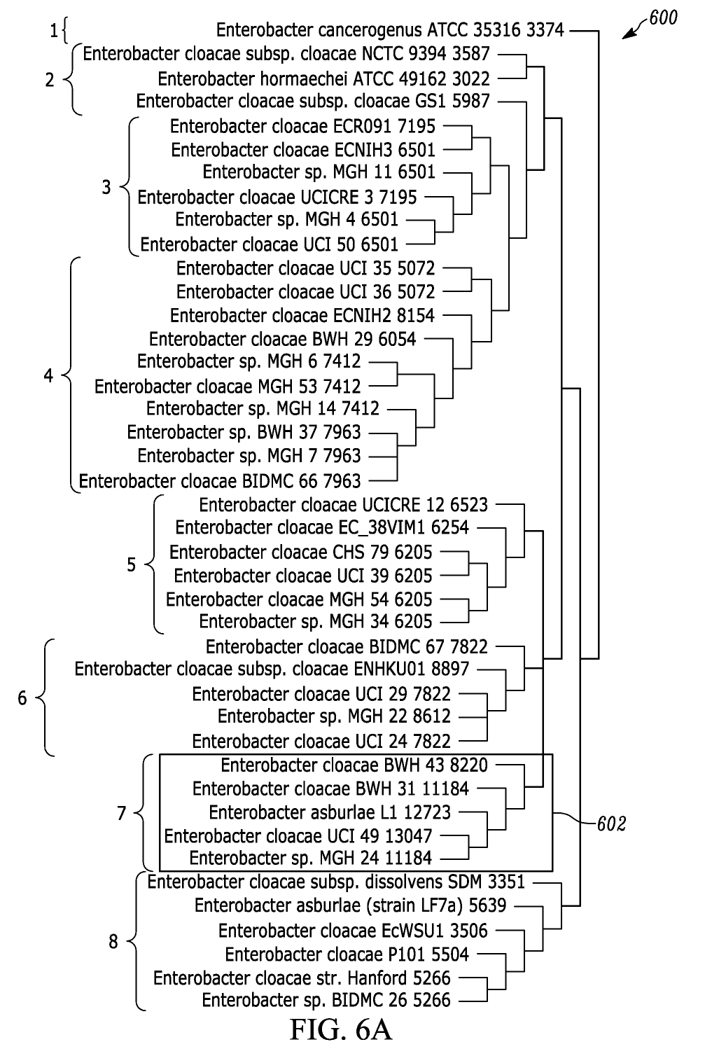

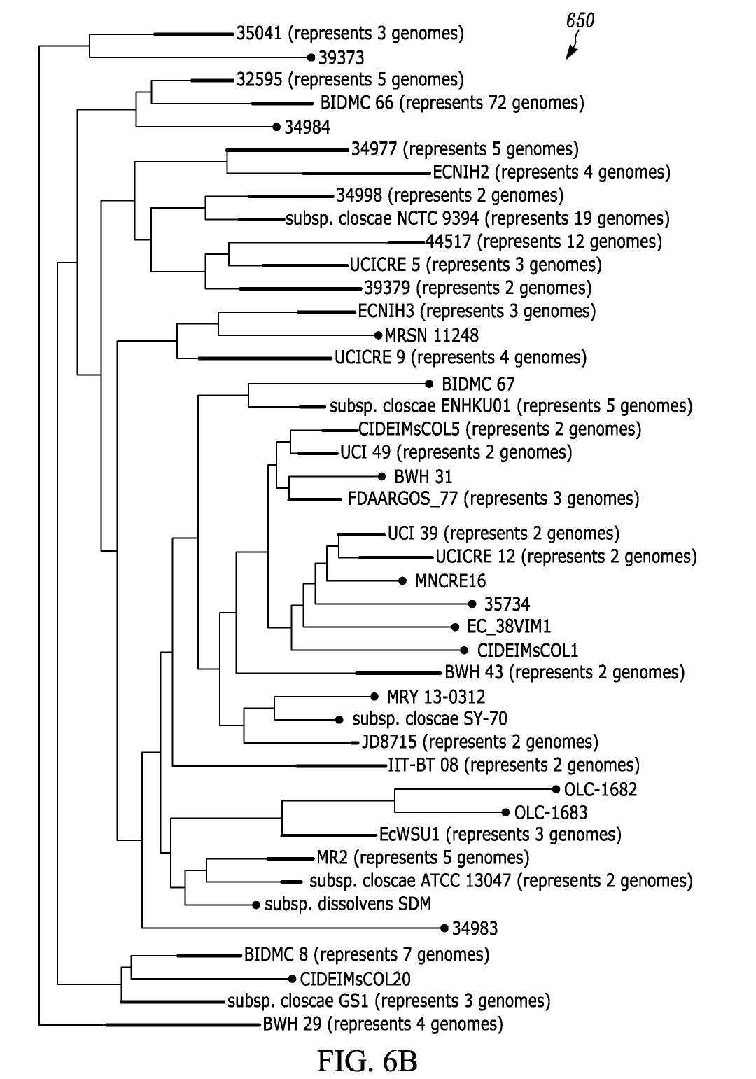

FIG. 6A shows a dendrogram 600 of the strains plotted in FIGS. 4A and 4B and FIGS. 3A and 3B. The dendrogram 600 is independent of the spectra, as it is based on shared ribosomal proteins in the database. In principle, the dendrogram 600 should be similar to other dendrograms that describe how strains are related to one another. For example, the dendrogram 650 presented in FIG. 6B was generated by “genomic blasting” and can be downloaded from http://www.ncbi.nlm.nih.gov/genome/1219. In practice, it is difficult to determine just how these dendrograms 600, 650 are related, as there are more strains in the NCBI database than in the TrEMBL database, and many of them have different names. Unfortunately, there are also significant differences in the naming of strains between one release of the TrEMBL database and the next release. This presumably reflects some recognition by the curators of the databases that the definition of species of many bacteria is unsatisfactory, and this appears to be especially true for Enterobacter. Therefore, it is not surprising that certain Enterobacter strain combinations are difficult to distinguish using the spectral databases matching methodologies that are used by Bruker and Biomerieux. One feature of the dendrogram 600 generated by the method of the present teaching, and shown in FIG. 6A, is that one can determine which combinations of strains are theoretically discernible by mapping to ribosomal proteins. Thus, the methods of the present teaching allow determination of approximately how much differentiation is possible, in keeping with the state of international understanding of just how different strains are related to one another. The identifiers in dendrogram 600 shown in FIG. 6A list the strain name, followed by the score from spectrum E3. Based on this dendrogram 600, the strains in FIG. 6A are divided into eight clades, as mentioned above, which are demarcated with brackets.

The score in plot 500 of FIG. 5A appears with different symbols on the plot according to these eight clades. For example, clade 7, highlighted by the rectangle 600 in FIG. 6A, consists of the five strains marked with x's, as shown in FIG. 5A. Note that according to FIG. 6A, one of the clade 7 strains is more divergent than the other four. This more divergent strain is the lowest scoring strain in FIG. 5A. Note that two of the six clade 8 strains correspond to the highest scoring strains from spectrum F3 according to plot 550 in FIG. 5B. Upon examination of the matching table, it appears that clade 8 is actually rather heterogeneous, which is why some members of this clade score are much lower in FIG. 5B.

One aspect of the present teaching is that it has been discovered that as annotations improve, it becomes easier to unravel which strain combinations are meaningfully different upon consideration of ribosomal subunit sequences only. Theoretically, the best proof of this discovery would be a careful study of multiple isolates from carefully annotated organisms. Ideally, different isolates should map to particular clades, and different isolates within each clade should tend to receive similar scores, as in FIG. 5A. In some embodiments of the present teaching, ribosomal proteins are differentially weighted according to how often they are mapped in representative spectra from certain clades of related organisms.

Another feature of the present teaching is that users can utilize standard statistical arguments to determine which spectra are meaningfully different from one another. This ability is independent of mapping to clades. To determine which spectra are meaningfully different using the method of the present teaching, multiple spectra are collected from two different isolates (isolates A and B). Ideally, these spectra should derive from separate colony extracts, in order to prevent sample preparation effects from driving the differentiation of A from B in the table. So long as there are consistent and significant differences in ranking and scoring between spectra from isolate A and isolate B, statistics can quantify the degree of meaningful strain differentiation, whether or not the database is correctly annotated for every protein sequence. Obviously, if the database has errors, there might well be corresponding errors in the assignment of strain A to clade X, or strain B to clade Y.

In some methods according to the present teaching that determine which spectra are meaningfully different, the score is defined as:

Score=log₁₀(% R)+log₁₀(% I)−log₁₀(ppm)

where % R is percent of ribosomal proteins matched, % I is the percent total intensity matched, and ppm is the RMS error (in parts per million) of the matched proteins. The minimum value for ppm is set to 100 to avoid very high scores for cases where a very small number of peaks is matched with smaller error than is feasible with the instrument. The score is calculated for all spectra of an isolate for each match. Then the mean, μ, and standard deviation, σ, for scores from each isolate for each match are calculated. The confidence that two matches are indistinguishable is given by:

P12=100×exp[−(μ₁−μ₂)²/2(σ₁₂−σ₂₂)].

For indistinguishable isolates, P12 is equal to 100 with a very small uncertainty and for distinguishable species P12 approaches zero. For related but distinguishable isolates, the confidence level is much smaller but may be greater than zero. In some methods of the present teaching, the score is based simply on log₁₀(% R)−log₁₀(ppm). In still other methods of the present teaching, the score is based simply on log₁₀(% R).

One feature of the methods of the present teaching is the ability to generate a dendrogram in the face of annotation artifacts. Many of the fine differences in dendrograms currently correspond to annotation artifacts, particularly, artifacts based on differences in the number of annotated ribosomal proteins. For example, strain discrimination might be dependent on inconsistent nomenclature for naming ribosomal proteins, or on inconsistent N-terminal extensions or deletions for some ribosomal proteins. In other cases, ribosomal protein sequences are polymorphic within a species. Certain strains should be distinguishable from one another for sound reasons, and in these cases there will be a substantial spread within a species between the highest and lowest scoring strains using the method of the present teaching. At present, careful attention is required to differentiate between meaningful strain differentiation and discrimination based on database artifacts. In a few cases in the TrEMBL databases, organisms are listed with names that are inconsistent with the majority of similarly named species, according to the pattern of shared ribosomal proteins. Mistakes in organism naming and in ribosomal protein naming and sequences should be eliminated in future releases. If the TrEMBL or the NCBI databases do not solve this problem, it is possible in principle to fix some of these problems at the SQLite level by writing a program that can correct many of the common database errors starting from the structure of ribosomes from well-studied organisms like E. coli.

In E. coli, there is compelling evidence that ribosomal protein L33 is quantitatively methylated. See, for example, Polevoda and Sherman (2007), “Methylation of proteins involved in translation”, Molecular Microbiology 65,590-606. The present teaching can be performed using a database in which L33 is always methylated, or using the standard database.

The L33 mass can be changed by 14 amu to correspond to a methyl group, and this improves scores, at least for many gram-negative bacteria (including the Enterobacter examples in FIGS. 4 and 5 above), using the methods of the present teaching. The method of the present teaching imports protein sequence information from a relational database in SQLite that is readily annotated using SQL update commands. This makes it possible to adjust annotations at the database level to correspond to known modifications. The methods of the present teaching allow protein sequences for certain protein classes to be modified to conform to what is known about the functionally active form of the protein. The methods of the present teaching will become more accurate as better techniques are developed to calculate mature ribosomal molecular weights from translated DNA sequences.

Another feature of the present teaching is the ability to adjust the weighting of various ribosomal proteins in calculating the score, based on various known biological characteristics. Some methods according to the present teaching combine the simplicity of matching only ribosomal subunits with the more comprehensive approach in which the entire proteome is matched. This can be readily accomplished by adjusting the weighting of each protein. The average mass of each protein can be calculated from the protein sequence. This protein sequence can be adjusted using the N-end rule (shared by most bacterial species) which removes N-terminal methionine from protein sequences if the second amino acid is one of the following six: ACGSTV. See, for example, Hirel et al (1989), “Extent of N-terminal methionine excision from Escherichia coli proteins is governed by the side-chain length of the penultimate amino acid”, Proc Natl Acad Sci USA. 86, 8247-51. Cysteines, however, are assumed to be fully reduced.

The mass of each protein is calculated, together with the mass of a doubly charged form of each protein, which is commonly observed in the 2-30 kDa mass region. Because scientists have studied some species more than others, some species like E. coli and S. aureus are represented several thousand times in this database. There appear to be about 1,600 genera represented in the databases, with ambiguity around the issue of just what constitutes a bacterial genus.

In some embodiments, ribosomal proteins are differentially weighted according to how often they are mapped in representative spectra from certain clades of related organisms. Certain proteins annotated as ribosomal are decremented in weighting because they are not well conserved across taxa, suggesting they are not the active ribosomal protein species. For example, some bacterial proteomes contain an L11 species that is much less homologous to other L11 molecules in related clades. It is likely that the less homologous L11 molecules are non-functional or contain sequencing errors, and therefore they should be weighted less than usual in deducing strain identity.

In some embodiments, proteins are differentially weighted within a clade according to how well represented the protein is within the clade. For example, certain proteins have been identified in every isolate so far sequenced from Klebsiella. In attempting to distinguish between Klebsiella strains, these proteins may be weighted either higher or lower based on conservation within Klebsiella. Other proteins may appear infrequently within the known sequences of an organism. The weighting of these proteins may be adjusted either up or down. Proteins may be annotated as a family using public annotations like Pfam, or by defining homologous sets of proteins based on shared C-terminal or N-terminal sequences. Most related series of proteins can be grouped by shared C-terminal hexapeptide sequences, even in the absence of standard bioinformatic mapping annotations like Pfam (data not shown).

It is expected that polymorphisms in certain protein families will be found to correlate well with correct strain identification. On this basis, the weighting of these protein families could be increased in subsequent searches. In contrast, protein families with polymorphisms that are never observed to correlate with correct strain identification could have weighting factors decreased.

The weighting of proteins may be weighted up or down depending on whether they are encoded on plasmids, in particular, plasmids that encode drug resistance factors. Similarly, proteins may be differentially weighted if they are nearby transposable elements, or phage proteins, as these regions of the genome are more likely to be unstable. Proteins directly association with transposition, plasmid tolerance, or phage metabolism may be weighted up or down depending on information gathered on expression of these proteins within a clade.

For example, an in-depth study of Staphylococcus may reveal that phage related proteins in general are poorly expressed. Accordingly, these proteins may be weighted down. Proteomic studies that deduce high protein abundance either from bottom up studies, or following purification of low molecular weight proteins can be used to adjust weighting factors within that clade. Protein weighting factors can be adjusted based on codon preference tables for the organism, or based on guanine-cytosine content (GC content) or on the difference between that GC content and the organism average.

The weighting of proteins may be weighted up or down depending on genomic distance from other proteins of key interest. For example, the mecA gene encodes a methicillin resistant penicillinase. The mecA protein itself is too large for easy MALDI identifications, but neighboring proteins on the genome are often of the appropriate size for MALDI identification (see Lau, 2014, “A rapid matrix-assisted laser desorption ionization-time of flight mass spectrometry-based method for single-plasmid tracking in an outbreak of carbapenem-resistant Enterobacteriaceae.”, J Clin Microbiol. 52:2804-12.). These proteins are more likely to correlate with mecA expression than proteins that are not nearby on the genome, and so these proteins could be weighted higher for identification of methicillin resistant penicillinase.

### EQUIVALENTS

While the Applicant's teachings are described in conjunction with various embodiments, it is not intended that the Applicant's teaching be limited to such embodiments. On the contrary, the Applicant's teaching encompasses various alternatives, modifications, and equivalents, as will be appreciated by those of skill in the art, which may be made therein without departing from the spirit and scope of the teaching.

What is claimed is:

1. A method for identifying microorganisms by MALDI-TOF mass spectrometry, the method comprising:
a) acquiring a MALDI mass spectrum of a microorganism;
b) detecting peaks in the acquired MALDI spectrum;
c) generating a peak list comprising mass and intensity from the detected peaks in the acquired MALDI spectrum;
d) acquiring a database of protein sequences deduced from DNA sequences for microorganisms with a computer;
e) generating with a computer a sub-database of ribosomal proteins from the protein sequences and their masses in the database;
f) matching masses of the detected peaks in the acquired MALDI spectrum to masses of the ribosomal proteins in the generated sub-database with a computer;
g) scoring the matches between the masses of the detected peaks in the acquired MALDI spectrum and the ribosomal proteins in the generated sub-database for each represented microorganism according to a percentage of intensity in the peak list that is matched (% I), a percentage of ribosomal proteins that can be accounted for (% R), and an intensity-weighted average mass error (ppm) for the matches to produce a score;
h) generating a peak list of accurate masses of matched ribosomal proteins in step g);
i) recalibrating the peak list comprising mass and intensity with the peak list of accurate masses of matched ribosomal proteins generated in step h);
j) identifying a microorganism with a highest score by sorting the scores using a computer; and
k) repeating steps f through j until a desired improvement in the recalibrated peak list or a validated identification is achieved.

2. The method for identifying microorganisms of claim 1 wherein the acquiring the database of protein sequences deduced from DNA sequences for microorganisms with a computer comprises downloading the database of protein sequences from a public internet site.

3. The method for identifying microorganisms of claim 1 wherein the acquiring the database of protein sequences deduced from DNA sequences for microorganisms with a computer comprises translating a database of DNA sequences from a public internet site into protein sequences with the computer.

4. The method for identifying microorganisms of claim 1 wherein the matching masses of the detected peaks in the acquired MALDI spectrum to masses of the ribosomal proteins in the generated sub-database comprises submitting the generated peak list comprising mass and intensity from the detected peaks in the acquired MALDI spectrum to a search algorithm executing on the computer.

5. The method for identifying microorganisms of claim 1 further comprising recording the peak list of accurate masses of matched ribosomal proteins in a computer database.

6. The method for identifying microorganisms of claim 1 wherein the scoring the matches between the masses of the detected peaks in the acquired MALDI spectrum and the ribosomal proteins in the generated sub-database for each represented microorganism according to the percentage of intensity in the peak list that is matched (% I), the percentage of ribosomal proteins that can be accounted for (% R), and the intensity-weighted average mass error (ppm) for the matches is determined from calculations of log₁₀(% R)+log₁₀(% I)−log₁₀(ppm) performed on the computer.

7. The method for identifying microorganisms of claim 1 wherein the scoring the matches between the masses of the detected peaks in the acquired MALDI spectrum and the ribosomal proteins in the generated sub-database for each represented microorganism according to the percentage of intensity in the peak list that is matched (% I), the percentage of ribosomal proteins that can be accounted for (% R), and the intensity-weighted average mass error (ppm) for the matches is determined from calculations of 2*log₁₀(% R)+log₁₀(% I)−log₁₀(ppm) performed on the computer.

8. The method for identifying microorganisms of claim 1 wherein the scoring the matches between the masses of the detected peaks in the acquired MALDI spectrum and the ribosomal proteins in the generated sub-database for each represented microorganism according to the percentage of intensity in the peak list that is matched (% I), the percentage of ribosomal proteins that can be accounted for (% R), and the intensity-weighted average mass error (ppm) for the matches is determined from calculations of log₁₀(% R)−log₁₀(ppm) performed on the computer.

9. The method for identifying microorganisms of claim 1 wherein the scoring the matches between the masses of the detected peaks in the acquired MALDI spectrum and the ribosomal proteins in the generated sub-database for each represented microorganism according to the percentage of intensity in the peak list that is matched (% I), the percentage of ribosomal proteins that can be accounted for (% R), and the intensity-weighted average mass error (ppm) for the matches is determined from calculations of log₁₀(% R) performed on the computer.

10. The method for identifying microorganisms of claim 1 further comprising computing a relative probability that a MALDI-TOF mass spectrum corresponds to an identified microorganism with a computer.

11. The method for identifying microorganisms of claim 10 wherein the relative probability that the MALDI-TOF mass spectrum corresponds to an identified microorganism is determined by calculating a mean, m, and a standard deviation, s, of the scores for each acquired mass spectrum with a computer.

12. The method for identifying microorganisms of claim 1 further comprising adding at least one ribosomal protein to the generated sub-database of ribosomal proteins from the DNA sequences.

13. The method for identifying microorganisms of claim 12 further comprising adding at least one of DNA binding protein HU or homologs to the sub-database of ribosomal proteins.

14. The method for identifying microorganisms of claim 1 where the % R term is calculated based on the number of proteins (R) that match within a particular mass range that is adjusted according to the peaks detected in the spectrum that is being matched.

15. The method for identifying microorganisms of claim 1 wherein the matching masses of the detected peaks in the acquired MALDI spectrum is extended to matching to all proteins in the proteome.

16. The method for identifying microorganisms of claim 1 wherein the matching the masses of the detected peaks in the acquired MALDI spectrum to masses of the ribosomal proteins in the generated sub-database comprises matching doubly charged forms of each protein.

17. The method for identifying microorganisms of claim 1 wherein the matching the masses of the detected peaks in the acquired MALDI spectrum to masses of the ribosomal proteins in the generated sub-database comprises performing differential weighting according to how often they are mapped in representative spectra from certain clades of related organisms.

18. The method for identifying microorganisms of claim 1 further comprising adjusting molecular weights of at least one of the masses of the ribosomal proteins to account for known stoichiometric modifications.

19. The method for identifying microorganisms of claim 18 wherein at least one of the known stoichiometric modifications comprises methylation.

20. The method for identifying microorganisms of claim 18 further comprising decrementing certain proteins annotated as ribosomal in weighting that are not well conserved across taxa.

21. The method for identifying microorganisms of claim 1 wherein proteins are annotated as a family using public annotations.

22. The method for identifying microorganisms of claim 1 wherein proteins are annotated using Pfam.

23. The method for identifying microorganisms of claim 1 wherein proteins are annotated by defining homologous sets of proteins.

24. The method for identifying microorganisms of claim 23 wherein proteins are differentially weighted according to C-terminal or N-terminal sequences.

25. The method for identifying microorganisms of claim 1 wherein proteins are differentially weighted within a clade.

26. The method for identifying microorganisms of claim 25 wherein proteins are differentially weighted within the clade according to how well represented the protein sequences are within the clade.

27. The method for identifying microorganisms of claim 25 wherein the differential weighting is adjustable up and down.

28. The method for identifying microorganisms of claim 25 wherein the differential weighting is adjustable up or down depending on whether the protein sequences are encoded on plasmids.

29. The method for identifying microorganisms of claim 28 wherein the plasmids encode drug resistance factors.

30. The method for identifying microorganisms of claim 25 wherein the differential weighting is adjustable down for protein families with polymorphisms that are never observed to correlate with correct strain identification.

31. The method for identifying microorganisms of claim 25 wherein the differential weighting is performed according to the protein sequence's position relative to transposable elements.

32. The method for identifying microorganisms of claim 25 wherein the differential weighting is performed according to the protein sequence's position relative to phage proteins.

33. The method for identifying microorganisms of claim 1 further comprising weighting proteins up or down depending on the protein sequence's association with transposition, plasmid tolerance phage metabolism, and information gathered on expression.

34. The method for identifying microorganisms of claim 1 further comprising weighting proteins up or down depending on proteomic studies that deduce high protein abundance.

35. The method for identifying microorganisms of claim 1 further comprising weighting proteins up or down depending on codon preference tables for the microorganism.

36. The method for identifying microorganisms of claim 1 further comprising weighting proteins up or down depending on guanine-cytosine content.

37. The method for identifying microorganisms of claim 1 further comprising weighting proteins up or down depending on how much the guanine cytosine content of the DNA sequence of the protein is different from the average guanine cytosine content for the microorganism.

38. The method for identifying microorganisms of claim 1 further comprising weighting proteins up or down depending on distance in base pairs from other proteins of interest encoded in DNA.

39. The method for identifying microorganisms of claim 1 wherein the matching masses of the detected peaks in the acquired MALDI spectrum to masses of the protein sequences in the generated sub-database comprises matching pairs of single and doubly charged masses.

40. The method for identifying microorganisms of claim 1 wherein the generating with the computer a sub-database of ribosomal proteins from the protein sequences and their masses in the database comprises preparing relational protein sub-databases from protein databases in which a combination of at least two of strain information, sequence information, and protein annotation are used to prepare the sub-database.

41. The method for identifying microorganisms of claim 1 wherein the matching masses of the detected peaks in the acquired MALDI spectrum to masses of the protein sequences in the generated sub-database comprises sorting with a computer by percent homology using a set of adjacent amino acids as an alignment key.

42. The method for identifying microorganisms of claim 1 further comprising modifying the protein sequences for certain protein classes to conform to known functionally active forms of the protein.
